(19) United States
(12) Patent Application Publication
(10) Pub. No.: US 2016/0023982 A1
(43) Pub. Date: Jan. 28, 2016

(54) METHODS FOR PRODUCING FREE FLOWING DICARBOXYLIC ACID CRYSTALLINE CROPS

(71) Applicant: BASF SE, Ludwigshafen (DE)

(72) Inventors: Christian DIENES, Wernersberg (DE); Matthias RAULS, Blieskastel (DE); Oliver BEY, Niederkirchen (DE); Matthias WEICKERT, Ludwigshafen (DE)

(73) Assignee: BASF SE, Ludwigshafen (DE)

(21) Appl. No.: 14/775,834

(22) PCT Filed: Mar. 14, 2014
(86) PCT No.: PCT/EP2014/055103

§ 371 Date: Sep. 14, 2015

Related U.S. Application Data
(60) Provisional application No. 61/786,953, filed on Mar. 15, 2013.

(30) Foreign Application Priority Data
Mar. 15, 2013 (EP) 13159503.5
Mar. 21, 2013 (EP) 13160355.7

(51) Int. Cl. C07C 51/43

(52) U.S. Cl. CPC C07C 51/43

(57) ABSTRACT

The invention relates to a process for producing free-flowing dicarboxylic acid crystals from an aqueous solution or suspension of the dicarboxylic acid in a crystallizer.

The invention relates to a process for producing free-flowing dicarboxylic acid crystals from an aqueous solution or suspension of the dicarboxylic acid in a crystallizer.

Dicarboxylic acids and especially adipic acid are important monomers for the preparation of polymers, in particular polyamides.

Dicarboxylic acids can be prepared, for example, by oxidation of cyclic alcohols, cyclic ketones or mixtures of these alcohols and ketones with oxidants such as concentrated nitric acid or air.

The most important dicarboxylic acid in terms of quantity is adipic acid which is obtained industrially from cyclohexane in two reaction steps. In the first step, cyclohexane is oxidized by means of air to form a cyclohexanolicyclohexanone mixture (anolone mixture). After unreacted cyclohexane has been separated off, the anolone mixture is oxidized by means of concentrated nitric acid.

The industrial oxidation of the cyclohexanolicyclohexanone mixtures is carried out using an excess of 40-70% strength by weight nitric acid, in particular concentrated (60% strength by weight) nitric acid, at from 40 to 90° C. and atmospheric pressure. Copper and vanadium salts serve as catalysts. The reaction output comprises the target product adipic acid (93-96 mol %) together with, as by- products, succinic acid and glutaric acid (4-5 mol %) and small amounts of monocarboxylic acids such as acetic acid. The reaction output is introduced into a degassing column into which air is blown from the bottom. The offgas taken off at the top of the column is worked up to give nitric acid, The bottom product is then dewatered in a second column to such an extent that the nitric acid content increases to 56% by weight. About 90% by weight thereof is recirculated to the oxidation reactor, while about 10% by weight is purified by crystallization from water, see DE-A-1 238 000.

It is also possible to oxidize cyclohexanol and/or cyclohexanone by means of air.

Recently, the preparation of adipic acid from adipic acid precursors which can be obtained from renewable raw materials such as sugar has become important. Thus, it is known that adipic acid can be obtained by hydrogenation of muconic acid (2,4-hexadienedioic acid). Furthermore, adipic acid can be obtained from glucaric acid.

Dicarboxylic acids having preferably from two to twelve carbon atoms, e.g. oxalic acid, malonic acid, succinic acid, maleic acid, glutaric acid, adipic acid, pimelic acid, suberic acid, sebacic acid, decanedicarboxylic acid and dodecanedicarboxylic acid, are crystalline compounds having melting points in the range from 98° C. to 185° C. They can decompose on heating, e.g. malonic acid into acetic acid and carbon dioxide. However, they can also, as in the case of, for example, succinic acid, form a cyclic anhydride or, as in the case of, for example, adipic acid, form a cyclic ketone (cyclopentanone).

For this reason, their purification by distillation has to be carried out under reduced pressure because of their lack of thermal stability. In many cases, purification is instead carried out by crystallization. Water frequently serves as solvent here. The solubility of the dicarboxylic acids in water decreases with increasing molecular weight.

To ensure ready processability and handling, the dicarboxylic acids are generally crystallized to form crystal powders (crystals). However, the crystals should not have an excessively low average crystal size distribution in order to reduce or prevent, for example, dust formation during handling.

However, such crystals often have the property of caking together to form larger crystal agglomerates during prolonged storage as bulk material. Relatively large transport and storage containers such as Big Bags or silos can therefore frequently only be emptied with considerable mechanical intervention for loosening caked crystals. This circumstance leads, for example in the further processing of adipic acid, to an undesirable additional outlay in terms of time and costs.

Adipic acid usually crystallizes from pure solutions in the form of thin platelets which have a large contact area and thus make good adhesion between adjacent crystals possible because of attractive interactions between the individual contact surfaces. Adipic acid crystals are described, for example, in R. J. Davey et al., J. Chem. Soc. Faraday Trans. 88 (23), 3461-3466 (1992). It has also been reported that the surface of pure adipic acid crystals is determined essentially by the crystallographic planes oriented in the {100} direction, whose physical properties are determined by the hydrophilic carboxyl groups present there. If two such (100) planes are brought into contact with one another, they can immediately stick weakly to one another by formation of hydrogen bonds. Such crystal surfaces are generally largely coated with a “monofilm” of water. Bringing two such water-coated surfaces into contact considerably strengthens the adhesion. The formation of such crystalline bridges is responsible for the above-described caking of the crystals.

A further disadvantage of such adipic acid crystals can be attributed to the crystal platelets formed being very thin. Thin crystal platelets break very easily during the production or processing process and in this way produce a generally undesirable proportion of fines. The associated broadening of the crystal size distribution is, firstly, frequently empirically associated with a deterioration in the flow behavior, and secondly the fines leads to dust formation during processing, as a result of which product losses can occur and it may be necessary to carry out complicated measures to ensure occupational hygiene.

The prior art describes a series of physical and chemical processes which allow the caking process to be suppressed. Thus, for example, in the storage of adipic acid in a product silo, small amounts of a dried gas are continuously passed through the silo. Since traces of moisture present are always largely carried away with this gas stream, the formation of intercrystalline bridges essentially does not occur and caking can be largely prevented in this way. However, this method has the disadvantage that it can be applied only with difficulty to transport containers, in particular not to Big Bags.

A further method of suppressing the strong intercrystalline adhesion is to coat the crystals with hydrophobicizing agents. Thus, for example, DE-A 1,618,796 describes a number of possibilities such as hydrophobicization of the surface of adipic acid crystals by application of monocarboxylic acids so as to prevent the formation of intercrystalline bridges. A disadvantage of these processes is that from 20 to 100 ppm of fatty acids has to be added to the adipic acid, and these remain in the product and thus make it unsuitable for applications having high purity requirements. In addition, this method requires an additional process step in the preparation of the adipic acid.

U.S. Pat. No. 5,296,639 describes a process for purifying adipic acid during the crystallization, in which the crystal morphology is modified so that the uptake of impurities during crystallization is reduced. For this purpose, caproic acid or selected surfactants such as sodium dodecylsulfate, sodium dodecylsulfonate or sodium dodecylbenzenesulfonate, for example, are added. A disadvantage of this process is that the additives typically have to be added in concentrations of from >100 ppm up to 3% in order to achieve the desired effect. The product is generally unacceptably contaminated as a result. In addition, there is the further disadvantage of the use of surfactants that in the case of accumulation due to internal recirculation of the solvent (generally water) they lead to foaming in plants, so that use in specific industrial processes is generally made more difficult or is not possible at all.

Subjecting solutions of dicarboxylic acids to crystallization with addition of at least one anionic polyelectrolyte having a molar mass of at least 2000 as crystallization aid is known from EP-A-0 968 167. The dicarboxylic acid crystals obtained here are significantly more compact at a larger average diameter.

However, the presence of additives is fundamentally disadvantageous. They display their effect only when they are adsorbed on the interface and therefore inevitably appear as impurities in the dicarboxylic acid crystals.

It is an object of the present invention to provide a process for producing free-flowing dicarboxylic acid crystals from an aqueous suspension of the dicarboxylic acid in a crystallizer, in which the concomitant use of additives can be omitted and readily storable and free-flowing crystals which do not tend to cake can nevertheless be obtained.

The crystals should preferably not only be larger than the known crystals, but should also not be obtained in the form of thin crystals having the crystallographic planes oriented in the {100} direction but instead in a more advantageous, compact crystal form.

The crystals should have good flow behavior and not lose their free-flowing nature on prolonged storage. In addition, they should not have any tendency to form fines and have a high purity.

The object is achieved according to the invention by a process for producing free-flowing dicarboxylic acid crystals from an aqueous solution or suspension of the dicarboxylic acid in a crystallizer, wherein, is used as crystallizer a stirred vessel which has a vertical, cylindrical tank having side walls and a bottom, means for introducing and discharging the solution or suspension, a guide tube arranged coaxially in the cylindrical tank and a blade stirrer which is arranged coaxially at the bottom of the tank and has a rotating coaxial shaft and stirrer blades and conveys the solution or suspension in a radial direction so that a flow of the solution or suspension in the manner of a loop reactor is established, where the circumferential velocity of the blade stirrer is from 0.5 to 6 m/s and the power input into the solution or suspension due to the blade stirrer is from 0.01 to 5 kW/m³, is used as crystallizer.

The term “solution or suspension” comprises solutions, suspensions and mixed solutions/suspensions. The terms “solution” and “suspension” preferably comprise these three meanings. Thus, the term “suspension” also encompasses solutions.

The term “free-flowing” refers to dicarboxylic acid crystals which do not have the form of thin platelets described at the outset but are instead three-dimensional crystal agglomerates which have a nonuniform surface structure and therefore do not tend to cake. Accordingly, they are free-flowing even after prolonged storage.

The term “aqueous solution or suspension” refers to a solution or suspension in which the solvent or suspension medium comprises predominantly water. The suspension medium preferably comprises at least 60% by weight of water, particularly preferably at least 80% by weight of water, in particular at least 95% by weight of water. Particular preference is given to using only water as solvent or suspension medium.

The term “flow of the suspension in the manner of a loop reactor” means that the suspension flows in the axial direction within the guide tube, likewise flows axially in the opposite direction between guide tube and side walls of the cylindrical tank and flows radially at the bottom of the cylindrical tank and above the guide tube. This results in loop flow of the suspension, based on a cross-sectional view of the cylindrical tank in the form of a vertical section, so that the suspension flows in the manner of a loop reactor. The term “flow of the suspension in the manner of a loop reactor” thus refers to loop flow of the suspension in a circuit into which suspension is introduced and from which suspension is discharged. There is no tubular loop but instead the loop is formed between cylindrical tank and guide tube. Relative to the vertical direction of the cylindrical tank, axial flow occurs through the vertical, cylindrical tank, with the exception of the upper and lower reversal points.

The term “blade stirrer arranged at the bottom of the tank” indicates that the transport direction of the blade stirrer in the direction of the bottom of the tank is restricted so that essentially or exclusively radial transport occurs at the bottom of the tank. The distance between the lower edge of the stirrer and the bottom of the tank is selected so as to be very small.

It has been found according to the invention that arrangement of a blade stirrer at the bottom of the tank results in advantageous crystal shapes if the circumferential velocity of the blade stirrer is from 0.5 to 6 m/s and the power input into the solution or suspension due to the blade stirrer is from 0.01 to 5 kW/m³.

This ensures, firstly, that a stable transport stream in which all suspended particles are moved upward from the bottom of the tank, are kept in motion and thus cannot cake at the bottom of the tank is obtained.

On the other hand, not too much shear energy is introduced into the suspension, so that the formation of crystal agglomerates is made possible.

The parameters according to the invention ensure a sufficiently large transport stream and a sufficiently small input of shear energy into the suspension.

Any suitable dicarboxylic acids which form a suspension in water can be used in the process of the invention. The dicarboxylic acid is preferably selected from among C₂₋₁₂-dicarboxylic acids, preferably C₄₋₈-dicarboxylic acids. The dicarboxylic acid is preferably aliphatic, linear and terminal or aromatic.

Examples of suitable dicarboxylic acids are terephthalic acid or isophthalic acid and also oxalic acid, malonic acid, succinic acid, glutaric acid, adipic acid, pimelic acid, suberic acid, sebacic acid, decanedicarboxylic acid and dodecanedicarboxylic acid. The dicarboxylic acids can be saturated, unsaturated or branched. In addition, the dicarboxylic acids can comprise further functional groups such as alkyl radicals having from 1 to 5 carbon atoms, hydroxyl, keto or halogen radicals.

Preference is given to using terephthalic acid, adipic acid or succinic acid for the pure crystallization of dicarboxylic acids. Particular preference is given here to adipic acid which has been prepared, in particular, by oxidation of cyclohexanol, cyclohexanone or mixtures thereof by means of nitric acid.

After removal of excess nitric acid, nitrogen oxides and water, it is preferably subjected to at least one crude crystallization, preferably two crude crystallizations. The adipic acid content of this prepurified adipic acid is preferably 95-99% by weight, more preferably 97-98% by weight (based on dry matter).

The cyclohexanol or cyclohexanone used for the oxidation by means of nitric acid can be prepared by oxidation of cyclohexane by means of air or oxygen, by hydrogenation of phenol or by hydration of cyclohexene.

Adipic acid which has been prepared by single-stage oxidation of cyclohexane by means of air or by hydrolysis of adipic diesters is also suitable.

Finally, it is also possible to use adipic acid which can be obtained from renewable raw materials, e.g. from muconic acid or glucaric acid.

The dicarboxylic acid suspensions used in the process are preferably obtained by the following steps:
a) oxidation of cyclic alcohols, ketones or mixtures thereof by means of nitric acid, oxygen or air to form corresponding dicarboxylic acids,
b) work-up of the oxidation output, with water and unconsumed oxidant being completely or partially separated off from the dicarboxylic acids formed,
c) at least one coarse crystallization of the dicarboxylic acids from water as solvent.

Crude adipic acid prepared by the abovementioned routes is preferably likewise subjected to at least one crude crystallization from water before the pure crystallization.

A crystallization generally occurs when a supersaturated solution of the dicarboxylic acid in a solvent is present. A supersaturated solution of a dicarboxylic acid can be produced in various ways:

Firstly, it is possible to bring about supersaturation of the solution by evaporation of solvent, under atmospheric pressure or under reduced pressure.

Secondly, a solution of the dicarboxylic acid can be produced at elevated temperature and this is then cooled to give a supersaturated solution which leads to crystallization.

It is also possible to combine the two measures, by cooling the solution by evaporation of solvent under reduced pressure to a temperature below the original solution temperature.

The pure crystallization of the dicarboxylic acids, in particular adipic acid, is preferably effected from water as solvent. It can be carried out batchwise or preferably continuously.

The dicarboxylic acids used for the pure crystallization, in particular adipic acid, preferably have a dicarboxylic acid content of 90-99% by weight after at least one crude crystallization.

The crystallization is preferably carried out at temperatures of from 30 to 90° C., more preferably from 40 to 80° C., particularly preferably from 50 to 70° C. The concentration of the aqueous dicarboxylic acid solution which is fed to the crystallizer is preferably from 20 to 70% by weight, more preferably from 30 to 60% by weight.

The residence time of the dicarboxylic acid suspension in the crystallizer is preferably from 0.25 to 8 hours, more preferably from 0.5 to 4 hours, particularly preferably from 1 to 3 hours.

It can be advantageous to place, for example, an adipic acid solution which comprises undissolved adipic acid in suspension as seed crystals in the crystallizer at the beginning of the crystallization. The temperature in the crystallizer is maintained, for example, at from 50 to 70° C. Crystal-free adipic acid solution is then fed in. Adipic acid suspension is continuously taken off via a product offtake at such a rate that the fill level of the crystallizer remains constant. As soon as the initially charged adipic acid suspension has been replaced, a new steady state equilibrium has become established.

The crystallizers used according to the invention are in their general form known from the prior art. On this subject, reference may be made, for example, to WO 2004/058377.

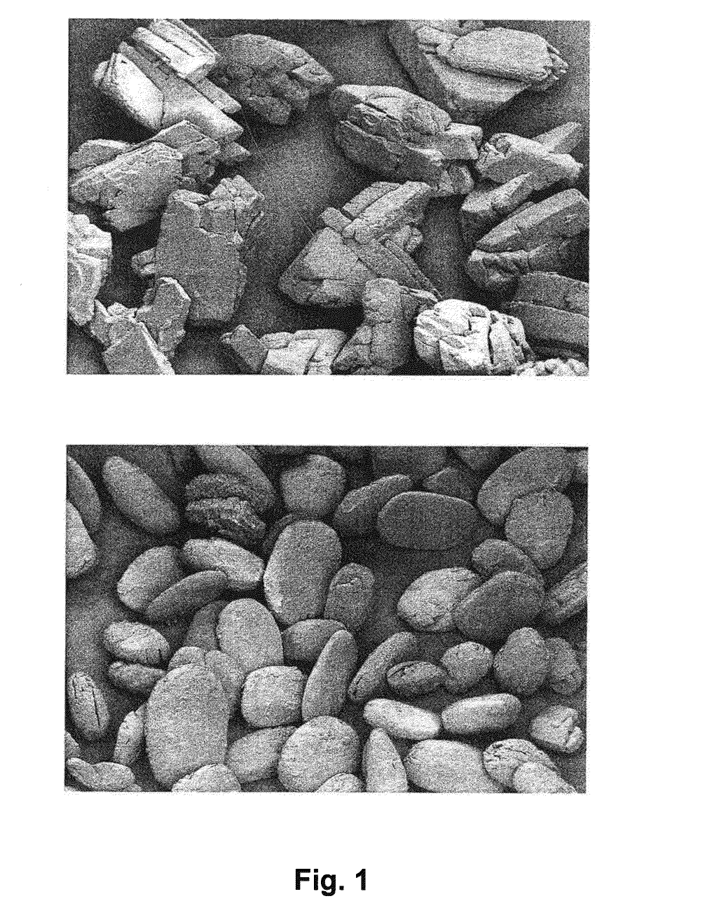

WO 2004/058377 A1 (DuPont, priority Dec. 16, 2002) describes an apparatus which is suitable for producing particles by precipitation or crystallization (FIG. 1 in WO 2004/058377). This apparatus can be configured so that biological products such as proteins and enzymes, small organic molecules such as pharmaceuticals, fine chemicals and inorganic materials such as mineral salts can be crystallized therein (page 1, lines 8 to 15). The apparatus depicted in FIG. 1 consists of an apparatus for crystallization, which comprises the following:
a) a vessel,
b) a radial stirrer which optionally comprises a cover plate and a bottom plate,
c) a guide tube which comprises a plurality of impingement plates which are arranged in such a way that a channel is formed between the guide tube and a side wall of the vessel, with the guide tube having a diameter which is about 0.7 times the diameter of the vessel.

Crystals of various sizes in the range from about 0.5 to about 3000 microns (page 6, lines 24 to 30) can be produced in the apparatus.

The crystallization of dicarboxylic acids, in particular of adipic acid, is not mentioned in

WO 2004/058377 A1. In a broad listing, mention is made merely of fatty acids which are monocarboxylic acids but not dicarboxylic acids.

WO 2004/058377 makes it possible to produce relatively large crystals having a relatively narrow size distribution or relatively fine crystals having a relatively narrow size distribution. Increasing the RPM often leads to finer particles, and the addition rate of the feed can be adapted in order to change the particle size (page 21, lines 22 to 25).

The size of the crystals can be varied by altering the chemical composition of the liquid streams, the stirrer speed (RPM) and the ratio of the various liquid streams relative to one another.

However, WO 2004/058377 A1 gives no information as to how the crystallization of dicarboxylic acids and especially adipic acid has to be carried out in order to achieve the object of the invention.

The configuration of the crystallizer used according to the invention will be described in more detail below.

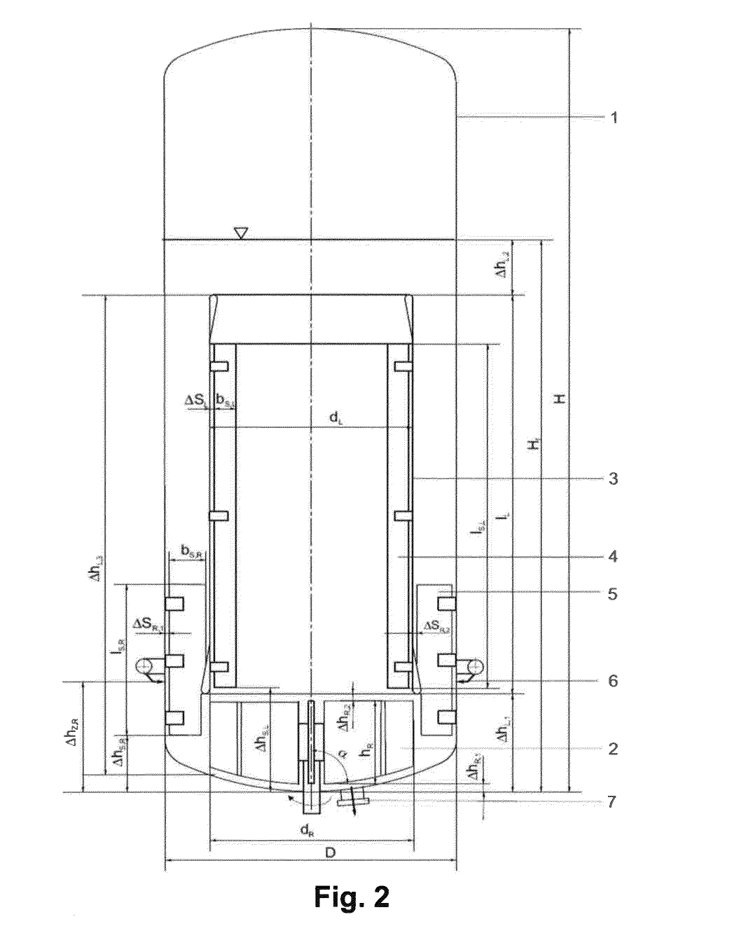

The parameters, definitions and abbreviations used below are shown in the accompanying FIG. 2.

FIG. 2 shows the schematic structure of a crystallizer used according to the invention in a cross-sectional view. The parameters mentioned in the following text are indicated in FIG. 2. FIG. 2 relates to a crystallizer used according to the invention with a radial stirrer arranged close to the bottom. The numerals 1 to 7 indicated at right in FIG. 2 have the following meanings:
1 Vessel
2 Radial stirrer
3 Guide tube
4 Guide tube baffle
5 Annular space baffle
6 Feed points for solution
7 Offtake

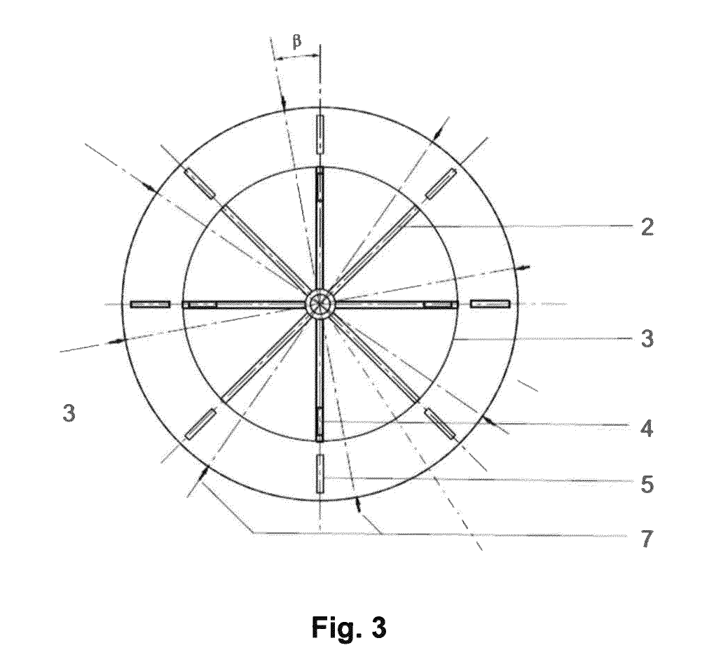

FIG. 3 schematically shows a horizontal cross-sectional view of the crystallizer of FIG. 2. The individual stirrer blades of the radial stirrer and the arrangement of the radial stirrer in the crystallizer can be seen here. The reference numerals 2 to 7 shown at the right-hand margin of FIG. 3 have the meanings indicated above.

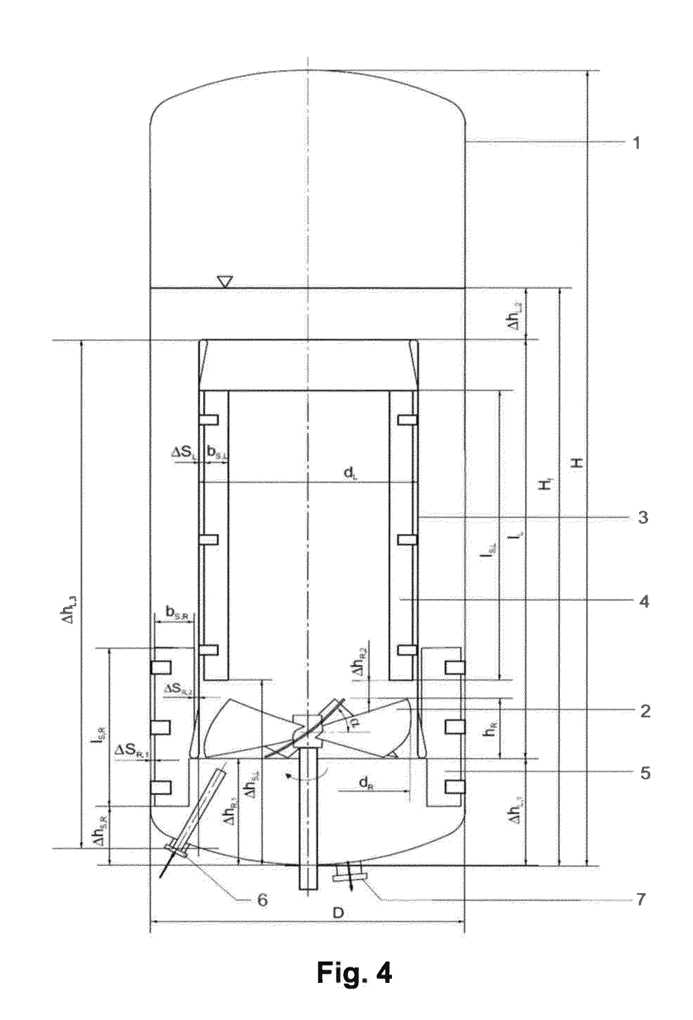

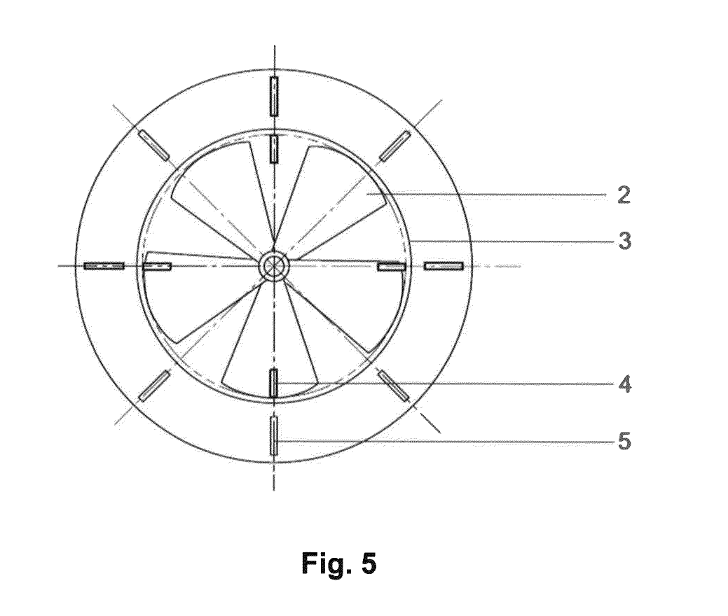

A possible configuration of an inclined-blade stirrer can be seen in FIGS. 4 and 5.

FIG. 4 shows a crystallizer which is not used according to the invention and in which an inclined-blade stirrer (actual stirrer) is not arranged close to the bottom but in the guide tube at a significant distance from the bottom. FIG. 4 shows a schematic cross-sectional view of the crystallizer and FIG. 5 shows a horizontal cross section through the crystallizer at the level of the inclined-blade stirrer. The arrangement of the inclined-blade stirrer in the crystallizer can therefore be seen from FIG. 5.

The inclined-blade stirrer per se can also be used in the process of the invention.

The reference numerals 1 to 7 used at the right-hand margin in FIGS. 4 and 5 have the following meanings:
1 Vessel
2 Axial stirrer (inclined-blade stirrer)
3 Guide tube
4 Guide tube baffle
5 Annular space baffle
6 Feed point for solution
7 Offtake

Preferred relationships according to the invention between the individual parameters can be taken from FIG. 2 which is true-to-scale in this respect.

In addition, FIG. 1 shows photographs of adipic acid crystals obtained by the process of the invention (FIG. 1 top) and conventional adipic acid crystals obtained by a process which is not according to the invention (FIG. 1 bottom).

As tank or vessel, a cylindrical basic shape which is closed off by a bottom and preferably a lid is selected according to the invention. As shapes of bottom and lid, preference is given to using dished plates, half-elliptical plates and three-center arched plates. However, flat and for the lower closure also conical plates are also possible. Particularly for the Anglo-American region, the shapes for bottom and lid can also be made according to the ASME (American Society of Mechanical Engineering) standard.

The aspect ratio H/D of the vessel, which is given by the ratio of the total height H of the vessel (including the bottom and the lid) to the vessel diameter D, is of particular importance. Preference is given to HID values in the range from 1 to 6, more preferably from 2 to 4, for the stirred tank or vessel.

The degree of fill of the vessel which is defined as the dimensionless ratio of the liquid fill level Hf to the total height H of the vessel is laid down as a function of the specific process conditions. A normal range for H_{f}/H is preferably from 0.5 to 0.9, more preferably from 0.6 to 0.8.

A plug-in tube, also referred to as guide tube, is arranged concentrically or coaxially in the vessel and serves to direct the circulation flow produced by the stirrer in a defined direction. An important design criterion by means of which the process parameters can be advantageously influenced is the ratio d_{L}/D of guide tube diameter d_{L} and vessel diameter D. Relatively small diameter ratios favor a dispersing function, while relatively large diameter ratios tend to result in gentle treatment of the material being stirred. Diameter ratios d_{L}/D are preferably in the range from 0.2 to 0.8, more preferably from 0.3 to 0.7.

The spacing between the lowest point of the bottom of the vessel and the lower edge of the plug-in tube Δh_{L,1} is selected so as to be large enough for the stirrer arranged in this intermediate space to be able to be operated with a safe distance to the bottom and to the plug-in tube. At the same time, this influences the deflection of the liquid flow between plug-in tube and annular space and thus the pressure drop in the loop. For industrial applications, the ratio of the distance from the bottom to the vessel diameter Δh_{L,1}/D is preferably from 0.1 to 0.6, more preferably from 0.15 to 0.5.

For stable operation of the guide tube apparatus, the supernatant liquid above the upper edge of the plug-in tube Δh_{L,2} is likewise advantageous in order to achieve favorable flow deflection and to keep the pressure drop caused thereby low. A favorable height of supernatant liquid can also favor intake of floating solid material or of foams. The height of supernatant liquid based on the vessel diameter Δh_{L,2}/D is preferably selected in the range from 0.05 to 0.5, preferably from 0.1 to 0.3. The length of the plug-in tube I_{L} is also determined by the configuration of the vessel height H, of the fill level H_{f}, of the bottom spacing of the guide tube Δh_{L,1} and of the height of supernatant liquid above the guide tube Δh_{L,2}.

It can be advantageous to provide baffles in the guide tube and/or between guide tube and side walls of the cylindrical tank. The baffles are important in order to break the tangential twist of the fluid flow brought about by the rotation of the stirrer and divert it into an axial flow direction. Firstly, the baffles should be arranged on the pressure side of the stirrer which, similarly to the case of pumps, is the side to which the stirrer pushes the fluid. In the case of the radial stirrer arranged between bottom of the vessel and plug-in tube, this is the region of the annular space between guide tube wall and vessel/tank wall. In the case of an axial stirrer which is arranged concentrically in the guide tube and whose transport direction in the guide tube is directed upward, the pressure side is the guide tube interior space above the propeller stirrer. If an axial stirrer conveys the fluid downward in the guide tube, the pressure side is the region between the lower edge of the stirrer and the bottom of the vessel. The baffling can then be arranged both in the diameter region of the guide tube and/or in the region of the annular space.

In the hydrodynamic experiments, it has been shown that suction-side baffling also improves the axial flow alignment and thus the transport effect. In the case of the radial stirrer arranged between the bottom of the vessel and guide tube, the suction side is the region of the guide tube interior space above the stirrer, likewise in the case of the axial stirrer which is arranged concentrically in the guide tube and transports downward in the guide tube. If the flow direction of an axial stirrer in the guide tube is directed upward, the suction side is the region below the stirrer.

Apart from the positioning, the dimensions and number of the baffles influences the baffling effect. The number of baffles per pressure and suction side is preferably from 3 to 12, more preferably from 4 to 8.

The width of the baffles b_{s/L} in the guide tube is expressed in dimensionless terms as a ratio to the guide tube diameter d_{L}. b_{s/L}/d_{L} is preferably the range from 0.05 to 0.5, more preferably from 0.1 to 0.35. The spacing of the baffles arranged in the guide tube between longitudinal side of the baffle and interior wall of the guide tube is normally ΔS,L=0.02×dL, but values from 0×dL to 0.1×dL are also possible. The length of the baffles in the guide tube IS,L can as a maximum correspond to the length of the plug-in tube IL and to ensure a sufficient baffling effect, should preferably be not less than half the diameter of the guide tube dL.

The distance between the guide tube baffles and the bottom ΔhS,L is, particularly in the case of the embodiment having the blade stirrer close to the bottom, preferably not less than the distance from the lower edge of the guide tube to the bottom of the vessel ΔhL,1 and not more than ΔhL,1+half the external diameter of the stirrer dR/2.

The width of the baffles in the annular space bS,R can as a maximum be the width of the annular space (D-dL)/2, which represents the clear spacing between guide tube and vessel wall. To give a sufficient baffling effect, the ratio of bS,R to the diameter of the vessel D should preferably be bS,R/D>=0.05×D, preferably bS,R/D>=0.1×D. The distance from the annular space baffle to the interior wall of the vessel ΔS,R1 is normally 0.02×D, but can also have values of from 0×D to 0.1×D. The distance from the annular space baffle to the interior wall of the guide tube ΔS,R2 can normally be 0.02×D, but, depending on the embodiment, can also be from 0×D to 0.1×D.

The length of the annular space baffles IS,R is guided by the need to achieve a sufficient baffling effect and should, similarly to the guide tube baffles, be not less than half the diameter of the vessel D. The maximum length is determined by the end of the annular space at the upper edge of the guide tube ΔhL,3. The distance from the annular space baffles to the bottom of the vessel ΔhS,R can preferably be in the range from 0×D to 1×D, preferably from 0.02×D to 0.5×D.

It has been found, according to the invention, that the size and in particular the shape (morphology) of the dicarboxylic acid crystals can be drastically improved by the speed of rotation or circumferential velocity of the stirrer (RPM) and the power input, preferably also by the stirrer type used.

To bring about turbulent mixing of the aqueous dicarboxylic acid solutions and suspensions in the crystallizer, the contents of the crystallizer should be intensively stirred.

The blade stirrer used in the crystallizer used according to the present invention has a rotating shaft and stirrer blades which are fastened thereto and can have any pitch relative to the stirrer shaft. Corresponding stirrer geometries are known per se and are described in detail in, for example, the documents indicated below. Here, any stirrer becomes a radial stirrer when it is arranged according to the invention close to the bottom.

EP-A-1 208 905 describes a stirred vessel for producing a suspension of solids in a liquid having a uniform concentration; here, the stirrer has turbine blades on a rotating shaft.

Textbooks and publications on stirrer design are, for example, B. K. Kipke, “Leistungsaufnahme und Fördermenge optimieren bei Leitrohrpropellern”, Maschinenmarkt, Würzburg 88 (1982) 52, Ekato-Handbuch der Rührtechnik” (1990); F. Liepe et al., “Rührwerke”, 1st edition 1998, publishing house of Fachhochschule Köthen.

The stirrer in FIG. 1 of WO 2004/058377 can have any shape as long as it makes the necessary circulation of liquid possible. Suitable stirrers are “radial flow impellers” which have an axial “flow propeller” or a “marine propeller”, a “double propeller” or a “multi propeller” at the top and/or at the bottom of the guide tube. The stirrer is preferably a “radial flow agitator”, preferably a “radial flow impeller” having at least one blade, a bottom plate and optionally a cover plate (page 10, line 27 to page 11, line 11).

The “radial flow impeller” can be modified in various places: the number of stirrer blades, the size of the blades, the blade pitch. In addition, the number of stirrer revolutions per minute (RPM) can be varied. The turbulence of the mixture which is desired in each case can be set via these parameters (page 11, lines 17 to 25).

The “impeller” preferably comprises the configurations shown in FIGS. 2, 3, 4 and 5.

The at least one blade of the stirrer according to WO 2004/058377, which can be used according to the invention, can have, for example, any shape as long as the contents of the crystallization apparatus are pumped through the apparatus at the necessary speeds. The height of the stirrer blades is typically about one-sixth of the agitator diameter. The width of the at least one blade varies with the blade pitch (page 12, lines 7 to 17). In general, the stirrer blades can have any pitch which brings about the necessary circulation in the apparatus. The blade pitch in WO 2004/058377 A1 is typically from about 45 to 65 degrees, preferably about 55 degrees (page 12, lines 7 to 17). The linear velocity of the slurry is from about 0.1 to about 1.8 meters per second, preferably about 0.9 meter per second (page 19, lines 12 to 14).

It can be seen from Römpp-Chemielexikon, 10th edition, volume 5, page 3876, keyword “Rühren”, that a considerable number of different stirrer types are used industrially. Thus, for example, fast-running stirrer types such as propeller, inclined-blade and disk stirrers or jet mixers are used for stirring in the low-viscosity range. Furthermore, blade stirrers are possible (not mentioned in Römpp).

As a result of the arrangement of the blade stirrer close to the bottom, any stirrer type used becomes a radial stirrer since the stream of suspension can only be conveyed outward since an axial flow direction is prevented by the bottom of the tank. The terms “close to the bottom” and “at the bottom of the tank” used according to the invention should be interpreted in this way.

It is therefore possible to use any suitable blade stirrer having a rotating coaxial shaft and stirrer blades. For example, the blade stirrer can be selected from among radial stirrers, inclined-blade stirrers, turbine stirrers, propeller stirrers, anchor stirrers, helical stirrers and screw-shaped stirrers.

The configuration and arrangement of the stirrer is important to achieve an optimal crystal morphology. The crystallization of adipic acid is preferably carried out using a radial stirrer which is located at the bottom of the tank/bottom of the vessel or between the bottom of the vessel and the lower edge of the plug-in tube and thus below the guide tube. A single-stage stirrer configuration is preferably selected, but division of the stirrer into a plurality of individual stages is also possible. In radial stirrers, the stirrer blades are arranged perpendicular to the horizontal plane at a pitch a of 90°. Typical representatives are disk or blade stirrers. The number of stirrer blades nRb can preferably be from 2 to 16, more preferably from 4 to 8. The stirrer height hR depends on the distance of the lower edge of the plug-in tube from the bottom ΔhL,1 taking into account a sufficient distance of the lower edge of the stirrer from the bottom ΔhR,1 itself and also a sufficient distance between upper edge of the stirrer and guide tube ΔhR,2. Relative to the vessel diameter D, the stirrer can preferably be designed with a height hR in the range from 0.1×D to 0.58×D. more preferably from 0.25×D to 0.5×D. Relative to the vessel diameter D, the values of ΔhR,1 and ΔhR,2 can preferably be in the range from 0.01×D to 0.3×D, more preferably from 0.03×D to 0.2×D. The spacings ΔhR,1 and ΔhR,2 can be different from one another. If necessary, the shape of the stirrer blades can be matched to the shape of the bottom in order to achieve a uniform distance ΔhR,1.

The diameter of the radial stirrer dR is determined by the diameter of the guide tube dL, but can also be made smaller or larger than this. dR is usually from 0.1×D to 0.98×D, preferably from 0.15×D to 0.9×D, more preferably from 0.3×D to 0.7×D.

Apart from the use of a radial stirrer having a pitch of 90° relative to the horizontal, further stirrer designs are also conceivable for arrangement close to the bottom underneath the guide tube. Pitches of the stirrer blades which are in the range from 25° to <90° relative to the horizontal are also possible. This geometry includes, in particular, axial stirrers such as inclined-blade stirrers, turbine stirrers and propeller stirrers. Apart from these, designs such as anchor stirrers, screw-shaped stirrers or helical stirrers are also suitable in principle. The direction of rotation of the stirrer drive can, seen from above, be either clockwise or counterclockwise.

Apart from the above-described arrangement of the stirrer underneath the guide tube, arrangement of an axial stirrer at the bottom of the tank in the guide tube is also possible, as an alternative, to bring about and influence crystal formation as desired. Stirrers used for this purpose are the abovementioned axial stirrers which preferably have a pitch in the range from 25° to 45° relative to the horizontal. The number of stirrer blades can preferably be from 2 to 12, more preferably from 3 to 8. Here too, the direction of rotation viewed from the top of the vessel can be either clockwise or counterclockwise. In combination with the blade pitch in (mathematically) positive or negative direction of rotation relative to the horizontal, the transport direction of the axial stirrer is therefore upward or downward in the guide tube. The distance of the lower edge of the stirrer from the bottom ΔhR,1 is preferably at least the distance from the lower edge of the guide tube to the bottom of the vessel ΔhL,1 and not more than the sum of ΔhL,1 and half the diameter of the stirrer dR/2.

To form the adipic acid crystals, a particular volume-based power input which ensures complete suspension of the solid particles and a stable circulating flow between guide tube and annular space has to be supplied by the stirrer. In the following, the volume-based power input is the ratio P/V of the stirrer power P and the fill volume V of the vessel. Power inputs are from 0.01 W/I to 5 W/I, preferably from 0.05 W/I to 2 W/I, in particular from 0.1 to 0.5 W/I or kW/m³. The power input and the stirrer or vessel size determine the circumferential velocity and the external diameter of the stirrer. The stirrer speed (circumferential velocity of the blade stirrer) necessary to form the compact agglomerates according to the invention is from 0.5 to 6 meters per second, preferably from 1 to 5 meters per second, more preferably from 1.2 to 4 meters per second, particularly preferably from 1.5 to 3.5 meters per second.

It is thus found that when blade stirrers are used, large dicarboxylic acid crystals in the form of compact agglomerates, which remain free-flowing for weeks after drying, are formed. The process of the invention has the advantage that in a conventional apparatus it achieves the stated object solely by use of an unconventional stirrer arrangement and appropriate circumferential velocity and appropriate power input, but without the aid of additives.

The compact crystals formed according to the invention remain free-flowing since they do not cake to form larger units, like the thin dicarboxylic acid platelets normally formed, because of their shape.

If, instead of a blade stirrer, an inclined-blade stirrer which has a pitch of more than 20 degrees and is installed centrally in the guide tube is used at the same energy input as in the case of the blade stirrer, significantly smaller dicarboxylic acid crystals in the form of thin platelets are formed. These platelets cake to form lumps which do not flow within a few weeks.

Small amounts of dicarboxylic acid suspension are taken off continuously via an offtake tube, so that the fill level in the crystallizer remains constant. The dicarboxylic acid crystals are, for example, separated off in a centrifuge and optionally dried.

To ensure that all particle size fractions are taken off uniformly in continuous operation, the variant of taking off suspension at the bottom of the vessel is preferably selected, but other variants for taking off the suspension, for example in the interior of the guide tube or in the region of the annular space, are in principle also possible.

If the offtake is located at the bottom of the vessel and a radial or axial stirrer arrangement between the bottom of the vessel and the lower edge of the plug-in tube, or a downward-transporting axial stirrer in the guide tube at the bottom of the tank according to the abovementioned installation conditions, is selected, the dicarboxylic acid solution fed in is preferably introduced into the annular space in order to avoid short circuit streams between point of introduction and outlet. Due to the upward-directed flow in the annular space and the subsequent downward flow in the guide tube, the volume elements introduced are necessarily conveyed at least once through the circulation region of the vessel before they can leave the crystallizer. The number of introduction points in the annular space can be from 1 to 100 and preferably corresponds to the number of baffles in the annular space.

The vertical position of the points of introduction ΔhZ,R should, viewed from the bottom of the vessel, preferably be at least ΔhZ,R=ΔhR,1+hR/2 and not more than ΔhZ,R=ΔhL,3 (end of the annular space at the upper edge of the guide tube), preferably from ΔhZ,R=ΔhR,1+hR and ΔhZ,R=ΔhR,1+2 hR, so that the feed can reliably be taken up by the upward-directed transport stream of the stirrer. The points of introduction can be distributed both in the vertical direction over the height of the annular space and in the horizontal direction over the circumference of the annular space. The position of the points of introduction should preferably be positioned, viewed in the horizontal plane in the direction of rotation of the stirrer, upstream of the baffles at an angle p in the range from 1 to 45°, preferably from 5 to 20°.

The invention is illustrated by the following examples.

### EXAMPLES

### Laboratory Crystallization of Adipic Acid

A cylindrical glass laboratory crystallizer (DN300) having a capacity of 22 I, and internal height of about 350 mm and an internal diameter of 300 mm and provided with a guide tube (DN200) having a diameter of 206 mm can be equipped with various stirrers. A 35% strength suspension of adipic acid in water is placed in the crystallizer and heated to 60° C. A solids content of about 23% is established due to partial dissolution of the crystals. These crystals initially present, which have an average size of 100-200 μm, serve as seed crystals. To carry out a continuous crystallization, a 35% strength, crystal-free solution of adipic acid at 82° C. is fed into this crystallizer from a heated reservoir. In order to keep the temperature in the crystallizer at a constant 60° C. at an amount of feed of 11.7 kg/h, the crystallizer is cooled via its walls. The fill level in the crystallizer is kept constant by periodically taking off small amounts of suspension via a bottom outlet valve. After operation of the experiment for 10 hours, the suspension originally present has been replaced except for less than 1% of the initial amount; a new steady-state equilibrium in respect of the particle size and shape has become established. The suspended crystals are separated off by centrifuging for 3 minutes on a mesh basket centrifuge at 600 g, freed of adhering mother liquor by rapid spreading on an absorptive filter paper and finally dried overnight at 60° C. in a vacuum drying oven.

### Example 2

In the above-described experiment, an eight-bladed blade stirrer having a diameter of 183 mm and a blade height of 80 mm and arranged at the bottom of the tank (about 20 mm above the bottom of the tank) is used and rotated at 112 RPM, corresponding to a circumferential velocity of about 1.07 m/s. The power input is about 0.5 W/I.

FIG. 2 and FIG. 3 schematically show the crystallizer used in the process.

The crystals obtained in the crystallizer depicted in FIGS. 2 and 3 by the process of the invention have an average size determined by laser light scattering of 1100 μm and display the shape of compact agglomerates. On storage in a closed screw-cap bottle (bed height 0.2 m), the crystals remain free-flowing for weeks. They are depicted in FIG. 1 at the top.

### Comparative Example 1

Instead of the blade stirrer, a five-bladed inclined-blade turbine (pitch 38°) having a diameter of 185 mm was used at a height of about 80 mm above the bottom of the tank and within the guide tube. At a speed of rotation of 112 RPM as in example 1, no crystallization can be carried out since the pumping power of the stirrer and the power input are not sufficient to produce stable flow and to suspend the seed crystals. In order to attain the same power input of 0.5 W/I as in example 1, a speed of rotation of 300 RPM is set, corresponding to a circumferential velocity of about 2.9 meters per second.

FIG. 4 and FIG. 5 schematically show the crystallizer and inclined-blade stirrer used in comparative example 1. The crystals obtained in the experiment were again measured and examined.

At the end of the experiment, the crystals have an average size of 450 μm and are in the form of thin platelets. Under identical storage conditions, the bed is significantly cohesive and moderately caked after only 24 hours, and after a few weeks it is severely caked in the form of large, hard lumps. They are depicted in FIG. 1 at the bottom.

### Batchwise Crystallization of Succinic Acid

The crystallizer described in example 1 for the continuous crystallization of adipic acid and depicted in FIGS. 2 and 3 is now charged with 9.6 kg of succinic acid and 20.4 kg of water. The 32% strength solution composed of these has a saturation temperature of 68.5° C. After dissolution of all crystals at 75° C., the crystallizer is cooled to 68.3° C. and seeded at this temperature with 96 g of succinic acid crystals, cooled by a further 0.5 K and stirred at this temperature for half an hour. The still fluid suspension is then cooled from 68° C. to 30° C. over a period of 3.5 hours, with cooling commencing slowly at 2 K/h at the beginning, then being increased slowly and the final temperature finally being achieved at a highest cooling rate of 16 K/h. The isolation and work-up of the suspension is carried out in a manner identical to the above-described procedure for adipic acid.

### Example 2

In a first experiment, an eight-bladed blade stirrer having a diameter of 183 mm and a blade height of 80 mm and arranged at the bottom of the tank (about 20 mm above the bottom of the tank) is used and rotated at 90 RPM. The power input here is only about 0.2 W/I. The crystals have an average size determined on a vibrating sieve of 1440 μm and display the shape of compact agglomerates. On storage in a closed screw-cap bottle (bed height 0.1 m), the crystals remain free-flowing for weeks.

### Comparative Example 2

Instead of the blade stirrer, a five-bladed inclined-blade turbine (pitch 38°) having a diameter of 185 mm is used at a height of about 80 mm above the bottom of the tank and within the guide tube, as shown in FIGS. 4 and 5. Here too, it is again not possible to achieve any stable flow and suspension of all crystals at the low speed of rotation of 90 RPM. To attain the same power input of 0.2 W/I as in the reference example, a speed of rotation of 245 RPM is set. At the end of the experiment, the crystals have an average size determined by laser light scattering of 700 μm and have the shape of strongly rounded platelets. Under identical storage conditions, the bed appears significantly cohesive after only 24 hours, and after a few weeks is moderately caked in the form of large lumps.

1.-14. (canceled)

15. A process for producing free-flowing dicarboxylic acid crystals from an aqueous solution or suspension of the dicarboxylic acid in a crystallizer, wherein a stirred vessel which has a vertical, cylindrical tank having side walls and a bottom, means for introducing and discharging the solution or suspension, a guide tube arranged coaxially in the cylindrical tank and a blade stirrer which is arranged coaxially at the bottom of the tank and has a rotating coaxial shaft and stirrer blades and conveys the aqueous solution or suspension in a radial direction so that a flow of the aqueous solution or suspension in the manner of a loop reactor is established, where the circumferential velocity of the blade stirrer is from 0.5 to 6 m/s and the power input into the solution or suspension due to the blade stirrer is from 0.01 to 5 kW/m³, is used as crystallizer and wherein the distance of the lower edge of the blade stirrer from the bottom of the tank ΔhR,1, based on the diameter D of the cylindrical tank, is from 0.01×D to 0.3×D.

16. The process according to claim 15, wherein the circumferential velocity of the blade stirrer is from 1 to 5 m/s.

17. The process according to claim 15, wherein the power input into the solution or suspension is from 0.05 to 2 kW/m³.

18. The process according to claim 15, wherein the cylindrical tank has a ratio H/D of height H to diameter D in the range from 1 to 6.

19. The process according to claim 15, wherein the ratio d_{L}/D of guide tube diameter d_{L} to diameter D of the cylindrical tank is in the range from 0.2 to 0.8.

20. The process according to claim 15, wherein the ratio Δh_{L,1}/D of the distance of the lower edge of the guide tube from the lowest point of the bottom of the cylindrical tank Δh_{L,1} to the diameter D of the cylindrical tank is from 0.1 to 0.6.

21. The process according to claim 15, wherein the blade stirrer is arranged below the guide tube.

22. The process according to claim 15, wherein the blade stirrer is selected from among radial stirrers, inclined-blade stirrers, turbine stirrers, propeller stirrers, anchor stirrers, helical stirrers and screw-shaped stirrers.

23. The process according to claim 15, wherein the dicarboxylic acid is selected from among C₂₋₁₂-dicarboxylic acids.

24. The process according to claim 23, wherein the dicarboxylic acid is aliphatic, linear and terminal or aromatic.

25. The process according to claim 23, wherein the dicarboxylic acid is adipic acid or succinic acid or terephthalic acid.

26. The process according to claim 15, wherein the dicarboxylic acid solutions or suspensions used in the process are obtained by a process comprising:
a) oxidizing cyclic alcohols, ketones or mixtures thereof by means of nitric acid, oxygen or air to form corresponding dicarboxylic acids,
b) working-up the oxidation output from step a), with water and unconsumed oxidant being completely or partially separated off from the dicarboxylic acids formed,
c) at least one coarse crystallization of the dicarboxylic acids from water as solvent.

27. The process according to claim 15, wherein the dicarboxylic acids of the dicarboxylic acid solutions or suspensions used in the process are produced or obtained from renewable raw materials.

28. The process according to claim 15, wherein the circumferential velocity of the blade stirrer is from 1.5 to 3.5 m/s.

29. The process according to claim 15, wherein the power input into the solution or suspension is from 0.1 to 0.5 kW/m³.

30. The process according to claim 15, wherein the dicarboxylic acid is selected from among C₄₋₈-dicarboxylic acids.
